(19) United States
(12) Patent Application Publication
(10) Pub. No.: US 2004/0006302 A1
(43) Pub. Date: Jan. 8, 2004

(54) Coaxial delivery device

(76) Inventors: Hassan Chaouk, Smyrna, GA (US); George Shengelaia, Atlanta, GA (US); Bruktawit Asfaw, Norcross, GA (US)

Correspondence Address: BIOCURE, INC., 2975 GATEWAY DRIVE, SUITE 100, NORCROSS, GA, 30071, US

(21) Appl. No.: 10/350,744

(22) Filed: Jan. 24, 2003

Related U.S. Application Data
(60) Provisional application No. 60/351,599, filed on Jan. 25, 2002.

(51) Int. Cl. A61M 3/02

(52) U.S. Cl. 604/40; 604/82

(57) ABSTRACT

Coaxial catheter delivery devices, methods to form coaxial microcatheters, and methods to deliver two part compositions, especially useful for formation of coaxial microcatheters for use in delivery of two part compositions to the neurovascular system.

### PRIORITY CLAIM

This application claims priority to U.S. Provisional application Serial No. 60/351,599, filed on Jan. 25, 2002.

### BACKGROUND OF THE INVENTION

The invention relates to coaxial catheter delivery devices, methods to form coaxial microcatheters and methods to deliver two solutions to a site in a body. The methods are especially useful for formation of coaxial microcatheters for use in delivery of two solutions to the neurovascular system. In one example, a device and method is used for delivery of a composition comprising prepolymers that polymerize in situ to form a hydrogel medical device.

Dual lumen catheters are used for a number of purposes. Generally, one is used when two components are to be delivered and it is desirable to deliver the components separately. For example, it may be necessary to prevent the components from mixing until they are delivered to the intended site. Dual lumen catheters can have side by side lumens or coaxial lumens.

Hydrogels are useful for a number of biomedical applications. Prepolymers that form hydrogels in situ are administered to the body in solution, whereupon they transform into the solid hydrogel. In situ forming hydrogels are especially useful for some applications, such as embolotherapy, tissue bulking, and drug delivery and are of several types. One type of in situ forming hydrogels is made from crosslinking macromers. Such macromers contain crosslinkable groups that can be crosslinked after administration (in situ) to form the hydrogel. See WO 01/68720 to BioCure, Inc. and U.S. Pat. No. 5,410,016 to Hubbell et al. for examples of such macromers.

WO 01/68720 describes a two part macromer system used to form a hydrogel in situ. Each of the two parts includes one part of a redox couple. When the two parts are combined, crosslinking (formation of the hydrogel) begins. It is sometimes preferable to delay mixing of the parts until they are delivered at the intended site of application. Premature mixing of the two parts can lead to unintended, premature formation of the hydrogel (and clogging of the catheter, for example).

In one embodiment disclosed in WO 01/68720, a side-by-side dual lumen catheter is used to deliver the macromer composition. One lumen delivers the reducing solution and the second lumen delivers the oxidizing solution. The macromer can be included in one or both of the reducing and oxidizing solutions. A disadvantage of side-by-side dual lumen catheters is that they are generally restricted in terms of size—they cannot be made below a certain diameter and maintain good flow characteristics or the needed flexibility to access tortuous or otherwise hard to reach sites—such as, particularly, neurovascular sites.

Another type of in situ forming polymer system is disclosed in U.S. Pat. No. 5,695,480 to Micro Therapeutics, Inc. In this system, a water insoluble polymer is injected dissolved in a non-aqueous solvent. The polymer precipitates into a solid as the solvent is displaced by aqueous body fluids.

U.S. Pat. No. 6,146,373 to Micro Therapeutics, Inc. discloses a catheter for use with the precipitating polymer. A first lumen is used to deliver the polymer dissolved in a solvent. The second lumen is used for delivery of an aqueous solution for controlling solidification of the polymer. This catheter is not suitable for use in neurovascular applications.

Accordingly, hydrogel biomaterials are desired for many applications and delivery systems and methods for their delivery and formation are needed. In particular, delivery systems and methods for delivery of prepolymers to form hydrogels in situ are needed for neurovascular applications. Moreover, coaxial microcatheters are needed for many applications.

### SUMMARY OF THE INVENTION

The invention relates to coaxial catheter delivery devices, methods to form coaxial microcatheters, and methods to deliver two solutions to a site in a body. The methods are especially useful for formation of coaxial microcatheters for use in delivery of two solutions to the neurovascular system. In one example, a device and method is used for delivery of a composition comprising prepolymers that polymerize in situ to form a hydrogel medical device.

### DETAILED DESCRIPTION OF THE INVENTION

The delivery devices and methods can be used for many applications; however, they are particularly useful for use as and for forming microcatheters for neurovascular use. “Microcatheter” means a catheter having a distal tip size of about 4 French or smaller.

I. Delivery Devices

The delivery devices include at least two catheters, a first, or outer, catheter and a second, or inner, catheter. The second catheter is positioned inside the first catheter to form a coaxial dual lumen catheter. The catheters are desirably used with a manifold, which provides for connection between the catheters and whatever type of containers the two solutions are delivered from—such as syringes.

The device can further include a syringe holder, into which the syringes can be placed so that delivery of the two solutions can be synchronized. A guidewire can be used, if desired, to aid in placement of the catheters.

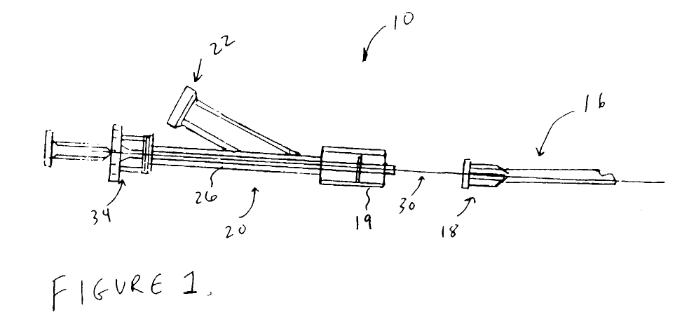

In one embodiment, illustrated by FIG. 1, the device 10 includes first catheter 16, which can be attached to the manifold 20 at its proximal end 18 via a luer adaptor 19, for example.

The manifold 20 includes a syringe adaptor 22 which provides connection (via a luer lock for example) between the interior space 26 of the manifold 20 (which leads into the first, outer catheter) and a syringe (not shown) for the first solution.

The second, inner, catheter 30 is sized so that it can be slid inside the first catheter 16. Furthermore, the second catheter should be sized to allow flow of a solution through the first catheter when the second catheter is in place. In other words, the second catheter should not fit too tightly within the first catheter.

The manifold 20 includes a second adaptor 34 to receive the second catheter 30. This can be a Tuohy-Borst adaptor, through which the second catheter can be inserted. The second catheter 30 is then pushed through the manifold and into and through the first catheter 16. Accordingly, the second solution delivered through the second catheter 30 does not contact the first solution delivered through the first catheter 16. A syringe (not shown) is fastened to the second catheter 30 for delivery of the second solution.

If desired, the first and second syringes are retained within a syringe holder (not shown) which allows synchronized delivery of the two solutions. The manifold would desirably be designed so that the syringes are aligned.

For placement of the delivery device within the vasculature at the intended application site, a guidewire (not shown) can be used.

The first catheter can be a commercially available catheter, such as a FasTracker 325 or Tracker 18 microcatheter. It should be of appropriate size to access the intended site of application. The outer diameter of the first catheter therefore can be of any size, so long as it is appropriate for the application. The presently disclosed device is particularly applicable for neurovascular applications or site selective applications which, in some cases, require microcatheters down to 1.6 Fr or smaller. The practical upper limit to catheter size is about 8 Fr.

In one example, the first catheter can be a Tracker 18, having an inner diameter of 0.021 inches. The second catheter can have an outer diameter of 0.012 inches, and an inner diameter of 0.009 inches. The space between the first catheter's inner diameter and the second catheter's outer diameter can vary in size.

The catheter delivery device is even more advantageous when a coaxial microcatheter below about 2.8 Fr is needed. The second catheter may be as small as about 0.7 Fr.

The second catheter may extend to the tip of the first catheter, may extend past the first catheter, or may not reach the tip of the first catheter, depending upon the design. If the second catheter does not extend as far as the first catheter, a mixing chamber is formed where the solution can mix prior to delivery. The device may include a stop mechanism to control the degree to which the second catheter can be inserted into the first catheter.

The first catheter can be made of typical catheter materials, typically a polymer such as, for example, polyurethane, polyethylene, silicone, or nylon. The second microcatheter can be made of a polymer but is desirably made of metal such as stainless steel or a binary nickel titanium alloy (nitinol). The second catheter can also be formed from standard catheter plastics but for use as a microcatheter is desirably formed from a metal, such as platinum, a platinum alloy, a nickel alloy, a titanium alloy, and some types of stainless steel (such as 316L stainless steel). Desirably, a binary nickel titanium alloy (nitinol) is used. Some plastics such as polyimide, polyethylene, polyurethane, and PTFE may be useful. The requirements for the fabrication material will depend upon the desired characteristics of the microcatheter, such as flexibility and strength, and the design parameters such as length and diameter. Desirably, medical grade superelastic nitinol is used.

In one variation, the first and/or second catheter can have one or more ports in addition to or instead of the end opening so that solution can be released over a larger area.

II. Methods

The methods for delivery of two solutions involve the use of two catheters. In a preferred method, the first (outer) catheter is positioned at the administration site, desirably using a guidewire. The second (inner) catheter is threaded through the first catheter (first removing the guidewire if one has been used).

The first and second catheters are connected to syringes or other dispensers holding the two solutions (as described above). The catheters may be part of a delivery device including a manifold and syringe holder, if desired. The method then involves delivering the two solutions.

The first catheter may be a commercially available catheter, such as a FasTracker 325 or Tracker 18 microcatheter, as described above in the device section. The second catheter may also be a commercially available catheter. Desirably, for uses requiring a microcatheter, the second catheter is a nitinol microtube, fitted with a syringe adaptor.

The method allows for placement of a coaxial microcatheter of a smaller size than would be possible with a prefabricated coaxial microcatheter. With a preformed coaxial microcatheter of the same diameter, a guidewire cannot be used since it cannot be threaded inside the inner lumen of the catheter.

Use with In Situ Polymerizing Prepolymers

In situ polymerizing prepolymer compositions can be used for a number of applications, including embolotherapy, tissue bulking, tissue sealing, drug delivery, etc. WO 01/68720 to BioCure, Inc. discloses an embolic composition wherein the prepolymers are crosslinked using a redox system. This composition can be delivered a variety of ways—one way is using two solutions, both containing prepolymer and one containing the oxidation agent and the other containing the reducing agent. For use in a microcatheter according to the invention, however, the viscosity of the solution delivered through the inner catheter must be low, as the i.d. of the catheter is so small. Accordingly, the method then involves delivering prepolymer solution through the first (outer) catheter; and initiator solution through the second (inner) catheter, wherein, when the prepolymer and initiator come into contact, the prepolymer forms a hydrogel medical device. The initiator can be either reductant or oxidant and the other of the pair is delivered with the prepolymer.

In an alternative embodiment, the initiator solution can also include prepolymer and can also optionally include contrast agent. Since-the viscosity of an initiator solution not containing prepolymer or contrast agent is much higher than one without these components, this embodiment is more appropriate for use with second catheters having a larger inner diameter.

The viscosities of the prepolymer solution and initiator solution can vary and should be appropriate for the size catheter being used. Generally, for a catheter ranging from about 3 Fr to 8 Fr, a viscosity of about 10 to 200 is appropriate. For a catheter ranging from about 1.6 to 3 Fr, a viscosity ranging from about 1 to 40 is appropriate. The solution can theoretically be any viscosity so long as it can be pushed through the catheter.

Other Applications

The devices and methods can be used for a number of applications—wherever it is desirable to deliver two solutions to a site. Solids can also be delivered when larger catheters are used. The precipitating polymer taught in U.S. Pat. No. 5,695,480 to Micro Therapeutics, Inc. can be delivered using this device and method, wherein the polymer is delivered through one catheter and an aqueous solution to aid in solidification is delivered through the other catheter.

The device and method could also be used for drug delivery, where it is desirable to deliver two drugs, or a drug and another component, such as embolic microspheres, at the same time but it is desirable for the two parts not to combine until delivery.

### EXAMPLES

The examples below serve to further illustrate the invention, to provide those of ordinary skill in the art with a complete disclosure and description of how the compounds, compositions, articles, devices, and/or methods claimed herein are made and evaluated, and are not intended to limit the scope of the invention. In the examples, unless expressly stated otherwise, amounts and percentages are by weight, temperature is in degrees Celsius or is at ambient temperature, and pressure is at or near atmospheric. The examples are not intended to restrict the scope of the invention.

### Example 1

### Rabbit Renal Artery Embolization Model

A New Zealand white rabbit was used to test the effectiveness of the method and device in embolizing the renal artery. The prepolymer had a PVA backbone (14 kDa, 12% acetate incorporation) modified with 0.45 meq/g N-acrylamidoacetaldehyde dimethyl acetal pendant polymerizable groups (about 6.3 crosslinks per chain). The prepolymer solution comprised 7% by weight prepolymer, 3% by weight AMPS, and 50% by volume Omnipaque 350, in 100 mM Acetate Buffer (pH=4.1) and 250 ppm hydrogen peroxide in DI water

The initiator solution included 2000 ppm Fe(II) lactate, 16.3 mM ascorbic acid, 20 mM acetate buffer in DI water.

The first catheter was a Tracker 325 and the second catheter was a nitinol tube, having an outer diameter of 0.012 and an inner diameter of 0.007.

Following induction of general anesthesia the right femoral artery was surgically exposed at the groin. After topical application of 2 ml 2% lidocaine the artery was ligated using a 2-0 silk suture and the arterial puncture was performed using a 22 G angiocath. A 0.014″ mandrel guidewire (Cook, Bloomington, Ind.) was advanced into the abdominal aorta. Then a sheath connected to a lactated Ringer's solution was placed into the femoral artery over the mandrel guidewire. The guidewire was removed and the animal was anticoagulated by 100 U/kg heparin. A FasTracker 325 microcatheter preloaded with a microguidewire was positioned into the abdominal aorta via the sheath. The microguidewire was removed and a midstream abdominal aortogram was then recorded using digital subtraction angiography (DSA) technique to evaluate the renal arteires. Then the road map image was taken to access the right renal artery. The microguidewire was placed back into the microcatheter. The right renal artery was accessed, the microguidewire was removed and 2.5 mg nitroglycerin diluted in 10 ml 0.9% NaCl solution administered via the microcatheter to the kidney. The right renal angiography performed. Then the kidney was irrigated by 10 ml 0.9% NaCl solution and the microcatheter was advanced closer to the organ. Under fluoroscopic control the second catheter was placed within the first catheter in such fashion that its tip was positioned 1-2 mm distally to the first catheter's marker. Through this delivery system 0.4 ml of each of the prepolymer solution and the initiator solution was administered to the right kidney over about 17 seconds. The delivery system was positioned in the abdominal aorta and the second catheter was removed. The first catheter was flushed by 5 ml 0.9% NaCl solution and follow-up abdominal angiography performed using DSA technique.

During injection of the prepolymer and initiator via the delivery system the entire renal artery was filled, without angiographic evidence of migration of the prepolymer or the formed hydrogel embolic agent to undesired areas.

### Examples 2-4

### In Vitro Examples

General

Gelation experiments were performed under flow conditions using a flow model, containing grooved channels in a branched arrangement, ranging in diameter from ⅛ to 1/32 of an inch that mimic an arterial venous malformation (AVM). The flow cell also contained a 0.37 inch circular void off the central flow channel to mimic an arterial aneurysm. The end of the flow model was fitted with two inlet ports to allow the insertion of a delivery system and the addition of a mobile phase (example: water, saline, PBS or serum). An outlet port was also present to allow the flow through of the mobile phase.

The prepolymer had a PVA backbone (14 kDa, 12% acetate incorporation) modified with 0.45 meq/g N-acrylamidoacetaldehyde dimethyl acetal pendant polymerizable groups (about 6.3 crosslinks per chain).

### Example 2

| | |
|---|---|
| first catheter: | Tracker 18 (Boston Scientific), 3.0/2.5 Fr, cut to |
| | 135 cm in length |
| second catheter: | Stainless steel, length = 150 cm, O.D. = 0.012 |
| | inches, I.D. = 0.009 inches |

Prepolymer Solutions:

(a) 7% prepolymer, 3% AMPS, 50% Omnipaque 300, 100 mM acetate buffer (pH 4.1) and 500 ppm H₂O₂.

(b) 12% Nelfilcon, 6% AMPS, 100 mM acetate buffer (pH 4.1) and 500 ppm H₂O₂.

Initiator Solutions:

(c) 41.5 mM Fe(II) lactate with 415 mM ascorbic acid

(d) 41.5 mM Fe(III) citrate with 415 mM ascorbic acid

Using a flow rate of 100 ml/min of water through the flow cell, and a delivery system as shown in FIG. 1, 1 ml of the prepolymer solution and 0.5 ml of the initiator solution were simultaneously injected. The following combination of solutions was employed: (a)+(c), (a)+(d), (b)+(c) and (b)+(d). In each case, a soft crosslinked hydrogel was formed in the central channel of the flow cell.

The experiments were repeated with the tip of the catheters inserted in to the circular aneurysm. On each occasion the circular void was filled with a soft, crosslinked hydrogel.

### Example 3

| | |
|---|---|
| first catheter: | Tracker 18 (Boston Scientific), 3.0/2.5 Fr, cut to |
| | 135 cm in length |
| second catheter: | Stainless steel, length = 150 cm, O.D. = 0.012 |
| | inches, I.D. = 0.009 inches |

Prepolymer Solutions:

(a) 7% prepolymer, 3% AMPS, 50% Omnipaque 350, 100 mM acetate buffer (pH 4. 1) and 500 ppm H₂O₂.

(b) 9% prepolymer, 3% AMPS, 48% Omnipaque 350, 100 mM acetate buffer (pH 4.1) and 500 ppm H₂O₂.

(c) 12% prepolymer, 6% AMPS, 1100 mM acetate buffer (pH 4. 1) and 500 ppm H₂O₂.

Initiator Solutions:

(d) 4000 ppm Fe(II) lactate with 83 mM ascorbic acid

(e) 41.5 mM Fe(II) lactate with 16.3 mM ascorbic acid

Using a similar protocol to that outlined in example 2, the following combinations of solutions were injected into the flow cell: (a)+(d), (a)+(e), (b)+(d), (b)+(e), (c)+(d) and (c)+(e). Using a flow rate of 100 ml/min, each injection produced a soft, cross-linked hydrogen when injected either into the central channel or the circular aneurysm.

### Example 4

| | |
|---|---|
| first catheter: | Tracker 18 (Boston Scientific), 3.0/2.5 Fr, cut to |
| | 110 cm in length |
| second catheter: | Stainless steel, length = 125 cm, O.D. = 0.012 |
| | inches, I.D. = 0.009 inches |

Prepolymer Solutions:

(a) 7% prepolymer, 3% AMPS, 50% Omnipaque 350, 100 mM acetate buffer (pH 4.1) and 250 ppm H₂O₂.

(b) 8% prepolymer, 3% AMPS, 50% Omnipaque 350, 100 mM acetate buffer (pH 4. 1) and 250 ppm H₂O₂.

(c) 9% prepolymer, 5% AMPS, 50% Omnipaque 350, 100 mM acetate buffer (pH 4. 1) and 250 ppm H₂O₂.

Initiator Solutions:

(d) 2000 ppm Fe(II) lactate with 16.3 mM ascorbic acid

(e) 2000 ppm Fe(II) lactate with 16.3 mM ascorbic acid and 50% Omnipaque 350

(f) 2000 ppm Fe(II) lactate with 16.3 mM ascorbic acid and 5% AMPS

(g) 2000 ppm Fe(II) lactate, 16.3 mM ascorbic acid, 50% Omnipaque 350 and 5% AMPS

Using a similar protocol to that outlined in example 2, the following combinations of solutions were injected into the central channel of the flow cell running with a flow rate of 70 ml/min. (a)+(d), (a)+(e), (a)+(f), (a)+(g), (b)+(f) and (c)+(f). On each occasion a soft crosslinked hydrogel was formed in the central channel of the flow cell.

Modifications and variations of the present invention will be apparent to those skilled in the art from the forgoing detailed description. All modifications and variations are intended to be encompassed by the following claims. All publications, patents, and patent applications cited herein are hereby incorporated by reference in their entirety.

What is claimed is:

1. A method for forming a coaxial catheter device to deliver two solutions to a site in a body, comprising the steps:
threading a first (outer) catheter to the site;
inserting a second (inner) catheter through the first catheter in a coaxial relationship.

2. The method of claim 2, wherein the method further includes the step of using a guidewire to position the first catheter at the intended site.

3. The method of claim 1, wherein the ends of the catheters are aligned or the first (inner) catheter extends up to 5 cm further distally than the second catheter so that a mixing chamber is formed.

4. The method of claim 1, wherein the first catheter is 4 Fr or less and the second catheter ranges in size from about 0.7 to 2.5 Fr.

5. The method of claim 4, wherein the second catheter is made of stainless steel or nitinol.

6. A method for delivering two solutions to a site in a body using a coaxial catheter delivery device, comprising the steps:
providing first and second solutions;
threading a first (outer) catheter to the site;
inserting a second (inner) catheter through the first catheter in a coaxial relationship;
delivering the first solution through the first (outer) catheter and the second solution through the second (inner) catheter.

7. The method of claim 6, wherein the first solution is a prepolymer solution, the second solution is an initiator solution, and when the prepolymer and initiator come into contact, they form a hydrogel medical device.

8. The method of claim 6, wherein the method further includes the step of using a guidewire to position the first catheter at the intended site.

9. The method of claim 6, wherein the ends of the catheters are aligned or the first (inner) catheter extends up to 5 cm further distally than the second catheter so that a mixing chamber is formed.

10. A coaxial catheter delivery device for delivering two solutions to a site in a body, comprising:
a first (outer) catheter;
a second (inner) catheter;
wherein the second catheter is inserted through the first catheter in a coaxial relationship and the first solution can be delivered through the first catheter and the second solution can be delivered through the second catheter.

11. The delivery device of claim 10, wherein the first catheter is 4 Fr or less and the second catheter ranges in size from about 0.7 to 2.5 Fr.

12. The delivery device of claim 10, wherein the second catheter is made of stainless steel or nitinol.

13. The delivery device of claim 10, further comprising a manifold to which the first catheter is attached at its proximal end and wherein the second catheter can be inserted through the manifold and into the proximal end of the first catheter.

14. The delivery device of claim 10, further comprising a syringe holder for holding syringes containing the prepolymer and the initiator, wherein the syringes are connected to the first and second catheters, respectively.
